(19) United States
(12) Patent Application Publication
(10) Pub. No.: US 2026/0346044 A1
(43) Pub. Date: Nov. 5, 2026

(54) PHOTOTHERAPEUTIC EYEWAER DEVICE

(71) Applicant: P.B.M Medical Technologies Ltd, Holon (IL)

(72) Inventor: Shlomo Lev, Holon (IL)

(21) Appl. No.: 19/488,863

(22) PCT Filed: Jun. 11, 2024
(86) PCT No.: PCT/IL2024/050579

§ 371 Date: Dec. 1, 2025

Related U.S. Application Data
(60) Provisional application No. 63/507,994, filed on Jun. 14, 2023.

(51) Int. Cl. A61N 5/06

(52) U.S. Cl. CPC A61N 5/0613; A61N 2005/0626; A61N 2005/0648; A61N 2005/065; A61N 2005/0659; A61N 2005/0663; A61N 2005/0666

(57) ABSTRACT

A phototherapeutic eyewear device is provided, the device includes a wearable eyewear frame and a triangular light guide. The triangular light guide includes a first plane and a second plane, perpendicular to the first plane, and a hypotenuse plane disposed at an angle with respect to the second plane. The triangular light guide is mounted on the frame such that the second plane is configured to face an eye of a wearer. The device further includes at least one light source configured to emit therapeutic illumination towards the first plane, such that the illumination enters inner portion of the triangular light guide. The device further includes a light diffusion coating on the hypotenuse plane configured to diffuse the illumination impinging on an inner surface of the hypotenuse plane, the light diffusion coating is further configured to reflect illumination impinging on the inner surface towards the second surface.

### FIELD OF INVENTION

The presently disclosed subject matter relates to an eyewear device including a light emitting source configured for phototherapeutic treatments of the eyes.

### BACKGROUND OF INVENTION

The eye is the sensory organ of the visual system that inserts the visual image through the cornea and the lens in projects it on the retina. The retina includes light-sensitive cells that convert light energy into electrical signals which are transmitted to the brain to decipher the observed image.

The retina contains several types of light-sensitive cells, some of which are called rods which are responsible for night vision and are more concentrated in the circumference of the retina. The density of the rods decreases towards the center of the retina to a point called the Blind Spot. At the center of the retina there is an area called the macula, where most of cells called cones are concentrated. There are three types of cones each of which respond specifically to three wavelengths of red, green and blue, which are the three primary colors that make up the color image.

All retinal celles are structurally supported by nerves celles also known as ganglion cells and bipolar cells as well as by layer of vascular blood vessel (choroid) that supports and supplies oxygen and nutrients to the retinal cells.

Every cell has a defined organelle called the mitochondria, whose main function is to create the energy needed for the cell and provide it in the form of molecules called ATP. Mitochondria are found in retinal cells at a large amount relative to other cell types, hence retinal cells regularly consume considerable amounts of energy compared to cells in other organs.

During a lifetime of any human the visual system decreases significantly and the retinal sensitivity to color vision gradually deteriorates. This decrease is caused by the decreased function of mitochondria which are produce less ATP especially for the rods and cones cells and therefore their activity weakens at a considerable rate.

Decreased or impaired function of the various types of retinal cells can be directly the result of a defect in the genetic sequence that causes, among other things, dysfunction of the mitochondria that cause a number of degenerative diseases called RP-Retinitis Pigmentosa. RP is a group of hereditary degenerative disorders which include difficulties in night vision, due to damaged rod and cone photoreceptor cells at the retinal region, which can lead to blindness.

The causes of decreased vision may start be caused by other factors, however eventually the decreased vision turns into damage to the retinal cells. This damage can come in several forms, which ultimately impair mitochondria and their function and eventually bring about the death of the cells.

The main diseases are:
AMD—Age related Macular Degeneration
DR—Diabetic Retinopathy
Retinopathy
Glaucoma
DME—Diabetic macular degeneration (DME)

Sometimes the damage to the retinal cells can be a result of an external factor such as, exposure or ingestion of various toxins, for example methanol, pesticides and chemical warfare agents.

Many studies have been performed on a variety of retinal diseases, and it was found that retinal irradiation at certain wavelengths, especially in the macula region, can stimulate the various retinal mitochondria to increase their activity to produce ATP and thus improve their function, stabilize their activity and slow the disease.

It is further noted that wavelength in the yellow spectrum, in particular 590 nm, was found to be beneficial in reducing VEGF protein, which are known to stimulate neovascular, which is a key factor in causing wet AMD. In other words, the use of this wavelength in case of a dry AMD reduces the productions of neovascular and prevents macular degeneration.

This low-energy radiation method is called photobiomodulation (PBM) and is performed in all wavelength ranges, but is mainly performed in the range between yellow 560 nm and near infrared (NIR) 1000 nm. It was proven that such radiation lowers the oxidative stress, increases NO (nitric oxide) release, increases ATP production, and decreases VEFG protein production improves cell durability.

It is known that the main mechanism by which the cell produces ATP, is carried out in mitochondria using the enzyme called Cytochrome C Oxidase (CcO). This enzyme contains a large protein complex with four centers of iron and copper atoms. In addition, this enzyme is responsible for regulating oxygen uptake (ROS) and the production of anti-inflammatory substances, such as NO.

The “action spectrum” of the enzyme is most effective around four peaks in the spectrum. One is around 620 nm, the other around 680 nm, the third around 760 nm and the last one is around 820 nm. Since the spectrum action is similar to the “absorption spectrum”, it can be said that the irradiation in the peak absorption regions intensifies the phase of electron transfer within the enzyme and thus the irradiation makes the electrons more available and hence increased ATP rate.

There are known treatments such as; Photobiomodulation—PBM which is performed in LED or low-level laser therapy (LLLT) diodes, including light source of a low energy laser or a laser diode of different wavelengths, especially in the red and infrared range.

An example of the use of a 670 nm wavelength using the PBM method is disclosed in a study entitled: Weeklong improved colour contrasts sensitivity after single 670 nm exposures associated with enhanced mitochondrial function, which can be found at: https://www.nature.com/articles/s41598-021-02311-1

This study describes direct irradiation of a red wavelength of 670 nanometers, performed in the morning using a flashlight that emits energy of 8 mW/cm2 for 3 minutes once a da. This causes an improvement in visual indices especially on the subject of Color Contrast Sensitivity—CCS observed 20% improvement in subjects of older ages.

Another example of the use of a 780 nm wavelength using the PBM method is disclosed in a study entitled: Low-Level Laser Therapy Improves Vision in Patients with Age-Related Macular Degeneration, which can be found at: https://www.liebertpub.com/doi/pdf/10.1089/pho.2007.2132.

This publication describes retinal irradiation of light in the infrared range (780 nm), for dry and wet AMD patients. The radiation was performed twice a day, twice a week at an energy of 7.5 mW/cm2 for 40 seconds (0.3 J/cm2).

The results were significant, and there was a marked improvement in visual acuity in about 95% without any side effects.

U.S. Patent No. US10596037B2 entitled Devices and methods for non-invasive multi-wavelength photobiomodulation for ocular treatments teaches a ophthalmic phototherapy device and associated treatment methods to expose an eye to selected multi-wavelengths of light to promote the healing of damaged or diseased eye tissue. The device includes a housing having an interior; an eyepiece disposed on the housing and configured and arranged for placement of an eye of the patient adjacent the eyepiece; a first light source producing a first light beam having a first therapeutic wavelength and disposed within the housing; a second light source producing a second light beam having a second therapeutic wavelength and disposed within the housing, where the second therapeutic wavelength differs from the first therapeutic wavelength by at least 25 nm.

US20180236258A1 teaches ophthalmic phototherapy devices and associated phototherapy methods for promoting healing of damaged or diseased eye tissue. An ophthalmic phototherapy device includes a light emitting mechanism for transmitting light of at least one preselected wavelength to the eye tissue. An ophthalmic phototherapy method includes directing light of at least one wavelength for a selected period of time to a portion of damaged or diseased eye tissue, whereby the light transmitted to the damaged or diseased eye tissue stimulates cellular activity in the eye tissue to promote healing.

Finally, US 2016/0067087 teaches a wearable ophthalmic phototherapy device and associated treatment methods to expose an eye to selected multi-wavelengths of light to promote the healing of damaged or diseased eye tissue. The device includes a frame having a front piece and two earpieces extending from the front piece; and at least one light source producing a light beam having a therapeutic wavelength and disposed within or on the frame. The devices can include multiple different light sources to produce light with therapeutic wavelengths that differs from each other by at least 25 nm. According to some examples discloses in this application, the front of the glasses are transparent and allow to see what is in front of the eye, while the radiation at different wavelengths comes from the inside of the frame and thus they come from different angles to the cornea.

The presently disclosed subject matter intends to overcome and combine the provision of energy in phototherapy treatment with high efficiency.

### SUMMARY OF INVENTION

There is provided in accordance with an aspect of the presently disclosed subject matter a phototherapeutic eyewear device, including a wearable eyewear frame, a triangular light guide having a first plane and a second plane, perpendicular to the first plane, and a hypotenuse plane disposed at an angle with respect to the second plane, the triangular light guide is mounted on the frame such that the second plane is configured to face an eye of a wearer. The phototherapeutic eyewear device further includes at least one light source configured to emit therapeutic illumination towards the first plane, such that the illumination enters inner portion of the triangular light guide; and a light diffusion coating on the hypotenuse plane configured to diffuse the illumination impinging on an inner surface of the hypotenuse plane, the light diffusion coating is further configured to reflect illumination impinging on the inner surface towards the second surface. The angle is configured such that illumination reflected by the light diffusion coating is directed towards eye of a wearer.

The phototherapeutic eyewear device can further include a light integrating box disposed between the at least one light source and the first plane, the integrating box is configured to increase illumination spread angle of the illumination and to guide isotropic illumination toward the first plane.

The light integrating box can be a rectangular portion made of reflective material integrally formed with the triangular light guide.

The light diffusion coating can be semitransparent configured to allow light impinging on an outer surface of the hypotenuse plane to enter inner portion of the triangular light guide.

The light diffusion coating can be configured to selectively reflect light within at least one predetermined wavelength region, and wherein the illumination from the light source is within the wavelength region.

The at least one source can include a plurality of light sources each of which is configured to emit therapeutic illumination of a predetermined wavelength.

The phototherapeutic eyewear device can further include a controller configured to control illumination of the plurality of light sources.

The phototherapeutic eyewear device can further include a controller configured to control illumination of the at least one light source, the controller is configured to selectively operate the at least one light source to a predetermined duration, and to control power of the illumination.

There is provided in accordance with another aspect of the presently disclosed subject matter a method for providing eye therapeutic illumination. The method includes providing phototherapeutic eyewear device, the device includes a wearable eyewear frame, a triangular light guide having a first plane and a second plane, perpendicular to the first plane, and a hypotenuse plane disposed at an angle with respect to the second plane, the triangular light guide is mounted on the frame such that the second plane is configured to face an eye of a wearer. The device further includes at least one light source configured to emit therapeutic illumination towards the first plane, such that the illumination enters inner portion of the triangular light guide, and a light diffusion coating on the hypotenuse plane configured to diffuse the illumination impinging on an inner surface of the hypotenuse plane, the light diffusion coating is further configured to reflect illumination impinging on the inner surface towards the second surface. The method includes activating the at least one light source with power and duration in accordance with a required eye therapy.

The light diffusion coating can be semitransparent configured to allow light impinging on an outer surface of the hypotenuse plane to enter inner portion of the triangular light guide, and wherein the activating includes determining power of duration in accordance with amount of light impinging on an outer surface of the hypotenuse.

The at least one source include a plurality of light sources each of which is configured to emit therapeutic illumination of a predetermined wavelength, and wherein the activation includes determining a light source to be activated in accordance with a therapeutic plan.

The plurality of light sources can include a first light source configured to illuminate light radiation in a first spectrum including wavelength of 590 nm, a second light source configured to illuminate light radiation in a second spectrum including wavelength of 670 nm, and a third light source configured to illuminate light radiation in a third spectrum including wavelength of 850 nm, the step of activation includes activating the first and second light sources simultaneously.

### BRIEF DESCRIPTION OF THE DRAWINGS

In order to understand the disclosure and to see how it may be carried out in practice, embodiments will now be described, by way of non-limiting examples only, with reference to the accompanying drawings, in which:

FIG. 1A is a perspective view of an eyewear device in accordance with an example of the presently disclosed subject matter;

FIG. 1B is a perspective view of an eyewear device in accordance with another example of the presently disclosed subject matter;

FIG. 2 is an exploded view of the eyewear device of FIG. 1B;

FIG. 3A is a perspective view of the optical arrangement of the eyewear device of FIG. 1A, in accordance with an example of the presently disclosed subject matter;

FIG. 3B is a side sectional view of the optical arrangement of FIG. 3A;

FIG. 4A is a side sectional view of the optical arrangement of FIG. 3A configured for allowing ambient view;

FIG. 4B is a side sectional view of the optical arrangement of FIG. 4A, including a correction prism;

FIG. 5 is a front view of an eyewear device in accordance with yet another example of the presently disclosed subject matter;

### DESCRIPTION OF THE PREFERRED EMBODIMENTS

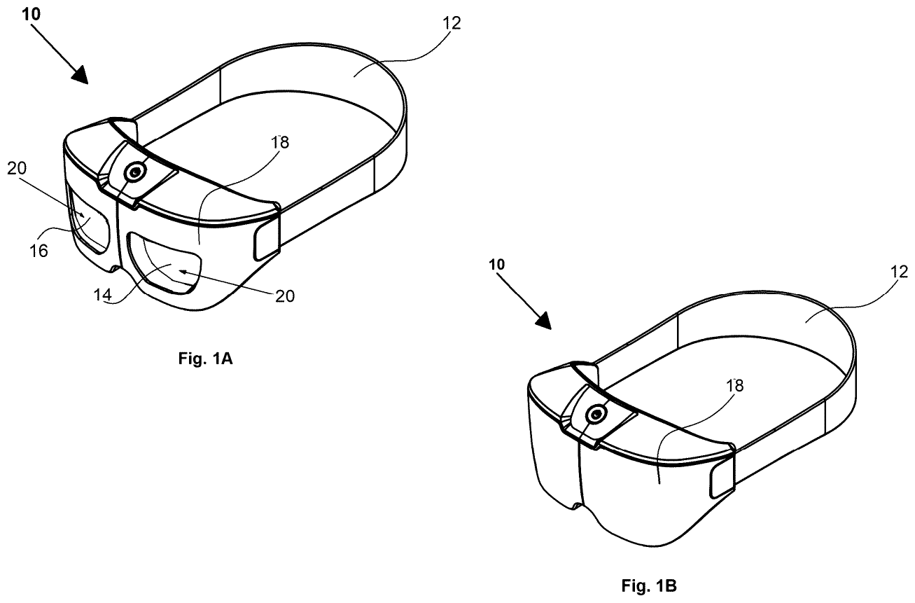

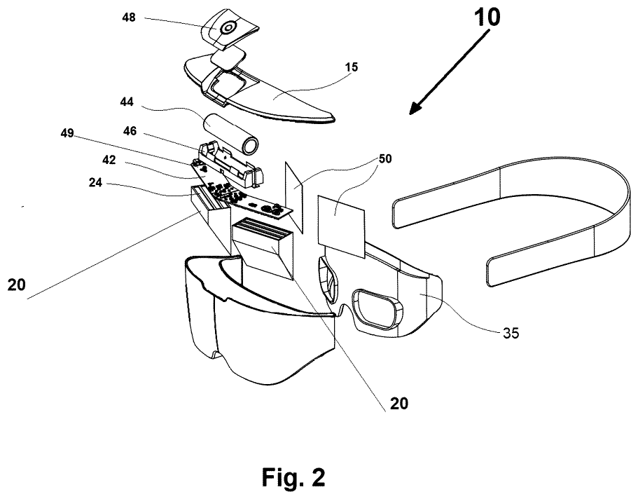

As shown in FIG. 1A, the presently disclosed subject matter is directed to a phototherapeutic eyewear device 10. The eyewear device 10 includes a wearable eyewear frame 18 and an optical arrangement 20, including one or more light sources 24 (shown in FIG. 2) configured to illuminate the eyes of a user with therapeutic illumination. The eyewear frame 18, according to the illustrated example, includes a strap 12, which can be replaced with a band or any other eyeglasses temples.

According to the illustrated example, the eyewear device 10 further includes optical apertures 14 configured for allowing vision therethrough. According to this example, the eyewear device 10 can be utilized for providing phototherapeutic to the eye of the user, while allowing the user to view through the eyewear device 10. This way, the user can wear the eyewear device 10 for an extended period of time while performing other daily activities, and receiving at the same time the required phototherapeutic treatment.

According to the Example of FIG. 1B, the eyewear device 10 can include a solid front without optical apertures. According to this example, the eyewear device is configured to provide therapeutic illumination without providing the user with view of the surroundings.

Referring now to FIG. 2, the eyewear device 10 includes a controller 42, and a power source 44 configured for providing electricity to the light sources 24. The eyewear frame 18 includes a cover 15 and a seat 46 for holding the power source 44, allowing thereby the power source 44 to be replaced. The eyewear frame 18 further includes an actuator 48, for activating the light sources 24.

The eyewear device 10 can further include adapting member 35 configured for facilitating mounting the eyewear device 10 over the face of the user. The adapting member can be made of foam or other flexible materials, and can be configured to block ambient light from reaching the eye of the user while the therapeutic illumination is directed to the eyes.

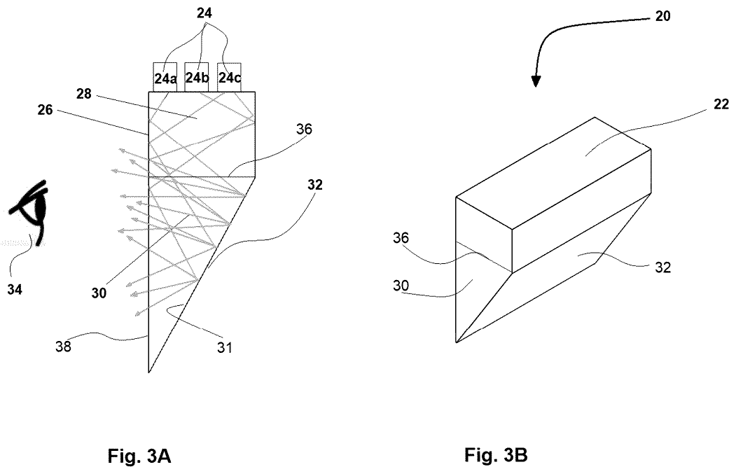

As shown in FIGS. 3A and 3B, the optical arrangement 20 includes a triangular light guide 30 having a first plane 36 and a second plane 38, perpendicular to the first plane 36, and a hypotenuse plane 32 disposed at an angle with respect to the second plane 38. The triangular light guide is mounted on the frame 18 such that the second plane 38 is configured to face the eye 34 of a wearer.

The light sources 24, which can include multiple light sources 24a, 24b and 24c, are configured to emit therapeutic illumination towards the first plane 36, such that the illumination enters inner portion of the triangular light guide 30.

According to the illustrated example, the optical arrangement 20 further includes a light integrating box 28 disposed between the light source 24 and the first plane 36 of the triangular light guide 30. The integrating box 28 is configured to increase the illumination spread angle of the illumination emitted from the light source 24 and to guide isotropic illumination toward the first plane 36. For example, the light integrating box 28 can be a rectangular portion having an upper surface 22 and a side surface 26 and can be made of reflective material configured to scatter light and to turn light rays isotropic, such that illumination is uniformly distributed in all directions.

Accordingly, light from the light source 24 entering the integrating box 28 strikes the internal walls 26, which reflect the light diffusely, scattering it uniformly throughout the integrating box 28.

Accordingly, light from integrating box 28 enters the triangular light guide 30 through the first plane 36, and the light spreads over a wider angle in comparison with the original beam angle of the light source 24.

It would be appreciated that the integrating box 28 can be integrally formed with the triangular light guide 30, precluding thereby any light fraction between the integrating box 28 and the first plane 36 of the triangular light guide 30.

The optical arrangement 20 further includes a light diffusion coating 31 on an inner surface of the hypotenuse plane 32. The light diffusion coating 31 is configured to diffuse the illumination impinging on the inner surface of the hypotenuse plane 32. The light diffusion coating 31 is further configured to reflect illumination impinging on the inner surface of the hypotenuse plane 32 towards the second surface 38.

The angle between the hypotenuse plane 32 and the second plane 38 is configured such that illumination reflected by the light diffusion coating 31 is directed towards eye 34 of a wearer. Moreover, the light diffusion coating 31 can be configured to selectively reflect light in the spectrum of the radiation emitted from the light source 24. In other words, the light diffusion coating 31 can be configured to selectively reflect light at least one predetermined spectrum, such that the spectrum having the therapeutic properties is evenly diffused and reflected toward the eye 34. This way, the eye is illuminated with isotropic light specifically in the spectrum, which is most efficient for to achieve the therapeutic goals.

According to an example, the spectrum emitted from the light source 24 is selected depending on the treatment required. A preferred wavelength that provides phototherapy benefits to retain cells and especially to cone cells is a wavelength of 670 nm, which is located at the edge of the visible spectrum. In this way, the interference of the phototherapeutic radiation with the vision through the eyewear device 10 is reduced or even completely precluded. Similarly, the wavelength can be in the infrared spectrum, for example, 850 nm, which is known to have a good impact on the cone cells of the retina.

According to an example, the light source 24 includes a first light source 24a configured to illuminate light radiation in a first spectrum including a wavelength of 590 nm, a second light source 24b configured to illuminate light radiation in a second spectrum including wavelength of 670 nm. The light source 24 can further include a third light source 24c configured to illuminate light radiation in a third spectrum, including a wavelength of 850 nm.

According to an example, the integrating box 28 can be configured for scattering and mixing light from two or more light sources 24a-24c. This way, the light radiation from the light sources 24a-24c is evenly distributed, providing isotropic illumination toward the first plane 36. It would be appreciated that the dimensions of the integrating box 28 are configured in accordance with the degree of required isotropic illumination taking into consideration the spectrum of each light source 24a-24c.

Similarly, the light diffusion coating 31 can also be configured to diffuse light in each of the spectrums of the light sources 24a-24c.

Furthermore, the eyewear device 10 can further include an auxiliary diffuser 50 (shown in FIG. 2) provided on an outer surface of the second plane 38. The auxiliary diffuser 50 can be configured to further evenly distribute the light radiation directed to the eye 34. The position of the auxiliary diffuser 50 on the outer surface of the second plane 38 allows diffusing the illumination from the light sources 24a-24c, regardless of the angle at which it reflects from the hypotenuse plane 32.

As indicated above, with reference to FIG. 1A, the eyewear device 10, according to one embodiment, can be configured to allow the user to view his ambient while using the eyewear device 10. To that end, the eyewear device 10 further includes optical apertures 14 configured for allowing vision therethrough.

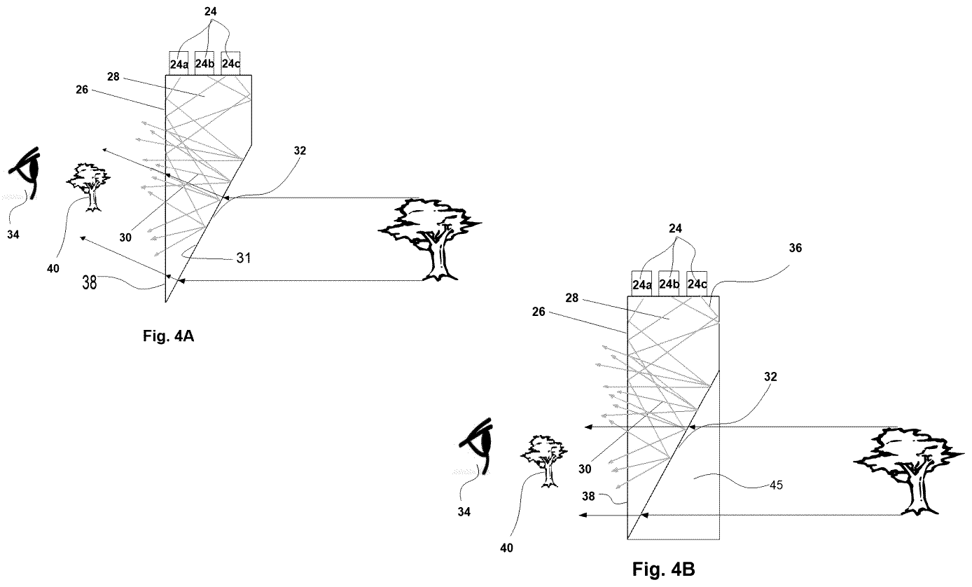

According to this example, as illustrated in FIG. 4A, the light diffusion coating 31 is semitransparent and is configured to allow light impinging on an outer surface of the hypotenuse plane 32 to enter an inner portion of the triangular light guide 30. This way, the triangular light guide 30 serves as an optical medium through which the user can view his ambient 40. This way, the user can wear the eyewear device 10 for an extended period of time while performing other daily activities, and receive the required phototherapeutic treatment at the same time. This way, the light radiation can be set to provide phototherapeutic, which requires long exposure.

The light diffusion coating 31 can be metallic coating such as gold, aluminum or silver and the thickness of the coating determines the reflecting rate of the light radiation from the light sources 24 a-24c. For example, for coating of 5 nm, the light diffusion coating 31 can transmit 50% of the light rays from the outside while reflecting 50% of the light radiation from the light sources 24a-24c. In addition, the power of the therapeutic light can be determined by controlling the power of the light sources 24a-24c.

As illustrated in FIG. 4A, due to the angle between the hypotenuse plane 32 and the second surface 38, outer light impinging on the hypotenuse plane 32 is refracted at such that the outer view through the triangular light guide 30 is distorted. As shown in FIG. 4B, in order to compensate for this distortion, a correction member 45 can be provided on the outer surface of the hypotenuse plane 32. For example, the correction member 45 can be a triangular prism configured to cancel out the distortion caused by the angle between the hypotenuse plane 32 and the second surface 38.

The eyewear device 10 can further include a controller 42 (shown in FIG. 2) for controlling the operation of the light sources 24a-24c, such as controlling the wavelengths of the light, and the duration of the radiation. This way, the controller 42 can be configured to provide the required therapeutic light radiation in accordance with a predetermined treatment protocol.

In particular, if the therapeutic illumination includes a combination of multiple wavelengths provided together or in series, the controller 42 can be configured to automatically initiate the therapeutic protocol. Furthermore, in case the eyewear device 10 is configured to allow ambient view, as shown in FIG. 1A, the controller 42 can be configured to initiate the therapeutic illumination at predetermined times of the day, such that the user can continue his daily activities while receiving the therapeutic illumination automatically.

According to an example the eyewear device 10 can include a transceiver 49 (shown in FIG. 2) allowing sending the controller 42 instructions related to the required treatment protocol, and for controlling the eyewear device 10. The transceiver 49 can be configured for short range communication, such as Bluetooth, and can be configured for communicating with a handheld device, such as a smartphone. The smartphone can be provided with a designated application to control the operation of the eyewear device 10. The application can be coupled to a remote server and can be further configured for communication with a remote controller, thereby allowing remote control of the eyewear device 10, such as via telemedicine services.

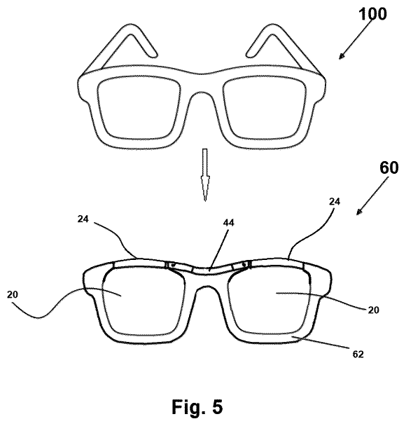

As shown in FIG. 5, according to an example, the eyewear device 60 can be a clip on eyeglasses, including a frame 62 and a pair of optical arrangement 20, similar to those described above. The eyewear device 60, according to this example, can a clip-on device, and can include a coupling member 44 for coupling to a frame of glasses 100. This way, the eyewear device 60 can be coupled to standard glasses, allowing therapeutic treatment. The coupling device 44 can be configured as a universal coupler, such that the eyewear device 60 can be mounted on any eyeglasses. The optical arrangements 20 are configured to allow vision therethrough and are further configured for illuminating the eye with therapeutic light radiation, such that the user can receive a therapeutic treatment while using the eyewear device 60 for vision. The optical arrangement 20, thus includes a light source 24 configured for emitting therapeutic light radiation for phototherapy of a part of a human visual system.

Those skilled in the art to which the presently disclosed subject matter pertains will readily appreciate that numerous changes, variations, and modifications can be made without departing from the scope of the invention, mutatis mutandis.

1. A phototherapeutic eyewear device comprising:
a wearable eyewear frame;
a triangular light guide having a first plane and a second plane, perpendicular to said first plane, and a hypotenuse plane disposed at an angle with respect to said second plane, said triangular light guide is mounted on said frame such that said second plane is configured to face an eye of a wearer;
at least one light source configured to emit therapeutic illumination towards said first plane, such that said illumination enters inner portion of said triangular light guide; and
a light diffusion coating on said hypotenuse plane configured to diffuse said illumination impinging on an inner surface of said hypotenuse plane, said light diffusion coating is further configured to reflect illumination impinging on said inner surface towards said second surface;
wherein said angle is configured such that illumination reflected by said light diffusion coating is directed towards eye of a wearer.

2. The phototherapeutic eyewear device of claim 1, further comprising a light integrating box disposed between said at least one light source and said first plane, said integrating box is configured to increase illumination spread angle of said illumination and to guide isotropic illumination toward said first plane.

3. The phototherapeutic eyewear device of claim 2, wherein said light integrating box is a rectangular portion made of reflective material integrally formed with said triangular light guide.

4. The phototherapeutic eyewear device of claim 1, wherein said light diffusion coating is semitransparent configured to allow light impinging on an outer surface of said hypotenuse plane to enter inner portion of said triangular light guide.

5. The phototherapeutic eyewear device of claim 4, wherein said light diffusion coating is configured to selectively reflect light within at least one predetermined wavelength region, and wherein said illumination from said light source is within said wavelength region.

6. The phototherapeutic eyewear device of claim 1, wherein said at least one source includes a plurality of light sources each of which is configured to emit therapeutic illumination of a predetermined wavelength.

7. The phototherapeutic eyewear device of claim 6, further comprising a controller configured to control illumination of said plurality of light sources.

8. The phototherapeutic eyewear device of claim 1, further comprising a controller configured to control illumination of said at least one light source, said controller is configured to selectively operate said at least one light source to a predetermined duration, and to control power of said illumination.

9. A method for providing eye therapeutic illumination, the method comprising:
providing phototherapeutic eyewear device, said device includes
a wearable eyewear frame;
a triangular light guide having a first plane and a second plane, perpendicular to said first plane, and a hypotenuse plane disposed at an angle with respect to said second plane, said triangular light guide is mounted on said frame such that said second plane is configured to face an eye of a wearer;
at least one light source configured to emit therapeutic illumination towards said first plane, such that said illumination enters inner portion of said triangular light guide; and
a light diffusion coating on said hypotenuse plane configured to diffuse said illumination impinging on an inner surface of said hypotenuse plane, said light diffusion coating is further configured to reflect illumination impinging on said inner surface towards said second surface;
activating said at least one light source with power and duration in accordance with a required eye therapy.

10. The method according to claim 9 wherein said light diffusion coating is semitransparent configured to allow light impinging on an outer surface of said hypotenuse plane to enter inner portion of said triangular light guide, and wherein said activating includes determining power of duration in accordance with amount of light impinging on an outer surface of said hypotenuse.

11. The method according to claim 9 wherein said at least one source include a plurality of light sources each of which is configured to emit therapeutic illumination of a predetermined wavelength, and wherein said activation includes determining a light source to be activated in accordance with a therapeutic plan.

12. The method according to claim 11 wherein said plurality of light sources includes a first light source configured to illuminate light radiation in a first spectrum including wavelength of 590 nm, a second light source configured to illuminate light radiation in a second spectrum including wavelength of 670 nm, and a third light source configured to illuminate light radiation in a third spectrum including wavelength of 850 nm, and wherein said activation includes activating said first and second light sources simultaneously.
